(19) United States
(12) Patent Application Publication
(10) Pub. No.: US 2026/0232242 A1
(43) Pub. Date: Aug. 13, 2026

(54) PET SANITATION AND WASTE MANAGEMENT SYSTEM

(71) Applicant: Pankaj Bhasin, Alexandria, VA (US)

(72) Inventor: Pankaj Bhasin, Alexandria, VA (US)

(21) Appl. No.: 19/534,347

(22) Filed: Feb. 9, 2026

Related U.S. Application Data
(60) Provisional application No. 63/755,555, filed on Feb. 7, 2025.

(51) Int. Cl. A61B 5/20; A01K 1/01; A01K 29/00; A61B 5/00; A61B 10/00; B08B 3/02; B08B 13/00; G01N 33/493

(52) U.S. Cl. CPC A61B 5/207; A01K 1/0107; A01K 29/002; A61B 5/7465; A61B 10/007; B08B 3/02; B08B 13/00; G01N 33/493; A61B 2503/40

(57) ABSTRACT

A pet sanitation and waste management system designed for efficient waste collection and disposal. It features an inner housing with a pan, a grate support, and a removable grate. Waste flows to a drain, aided by a waste pump connected to a waste tube. A wash pump linked to a wash tube with nozzles dispenses antibacterial cleaning solution, flushing waste into a removable waste container. The system includes an outer housing with a guard and a button to activate the pumps. Wash and waste containers feature one-way valve caps for secure coupling. The grate has an antibacterial, anti-slip rubber coating for hygiene and safety. This user-operated system ensures effective cleaning and odor control.

### CROSS REFERENCE TO RELATED APPLICATIONS

This application claims benefit from currently pending U.S. Provisional Application No. 63/755,555 titled “Pet Sanitation and Waste Management System” and having a filing date of Feb. 7, 2025, all of which is incorporated by reference herein.

### FIELD OF THE INVENTION

This technology pertains to the field of pet care, specifically targeting sanitation solutions for canines. It involves a platform integrating mechanical and fluid dynamic components designed to manage waste and maintain cleanliness, effectively minimizing contact with waste materials and enhancing hygiene in residential environments.

### BACKGROUND OF THE INVENTION

Current technologies that relate to systems and methods for managing pet sanitation generally relate to outdoor urination, urine pads, or litter boxes. These technologies generally present several challenges. For example, outdoor urination are dependent on the owner's availability to take the pet outside and may not be feasible in certain weather condition or living situations. Indoor pads, on the other hand, require frequent replacement and can create unpleasant odors, while also posing a risk of spillage or leakage. These types of manual systems require frequent user intervention, presenting a significant inconvenience and potential health risk due to exposure to waste.

Furthermore, automated systems currently available often lack comprehensive integration of components necessary to ensure thorough cleanliness and are prone to malfunction due to inadequate sensor technology or improper waste flow management. Such drawbacks can result in waste residue accumulation, offensive odors, and insufficient disinfection, compromising the system's intended purpose. Many automated canine hygiene systems on the market also fail to address the problem of minimal contact between the pet's paws and urine, a significant hygiene concern in controlling the spread of contaminants. Maintenance and cleaning of these systems can be cumbersome, often requiring disassembly or extensive manual effort, thereby reducing user convenience and satisfaction.

What is needed is an integrated sanitation platform for pets that addresses these deficiencies by combining mechanical and fluid dynamic components in a cohesive manner. Such a solution should efficiently manage waste while ensuring minimal contact between the canine and contaminants, utilizing sensors for responsive operation and incorporating automated cleaning mechanisms to sustain hygiene without requiring frequent user intervention. Enhanced user convenience and system reliability should be prioritized to enhance domestic usability and streamline the maintenance process.

### SUMMARY OF THE INVENTION

The present invention aims to provide a pet sanitation and waste management system that efficiently collects and disposes of a pet's waste. The system comprises an inner housing with a first and second section, a support grate, a waste pump, a wash pump, an outer housing, and a button coupled to a power support. The first inner section includes a pan with a grate support and a plurality of nozzles, designed to direct the pet's waste towards a drain. The second inner section houses a waste receptacle and a wash receptacle, both of which can be removably coupled to a waste container and a wash container respectively.

Another objective of the invention is to provide a system where the support grate has a plurality of channels and holes, allowing for the pet's waste to freely flow to the pan base. The power support can be coupled to the second inner section and can be supported on at least one side by a guard. A further aim of the invention is to provide a system where the outer housing has an open end and a closed end, with the closed end forming an outer housing base surrounded by one or more walls extending towards the open end. The guard can extend from a lower end to an upper end and can be removably or permanently coupled to the open end and/or the first inner section and/or the second inner section.

Yet another objective of the invention is to provide a system where the outer housing has at least two wheels coupled to it with a pull handle on at least one side of the walls. The outer housing also includes a switch and power adapter port, allowing the user to turn the system on and off. The invention also aims to provide a system where the waste container and the wash container have a seating cap with a one-way valve in its bottom, allowing fluid to flow in or out of the containers. The wash container can contain a wash fluid, which can be an antibacterial and scent cleaning solution.

A further aim of the invention is to provide a system where, when the button is pushed, the wash fluid is pumped from the wash container and through the wash tube, spraying the pan base and flushing the pet's waste down the drain and into the waste container. The grate can have a rubber coating providing an antibacterial and anti-slip coating. These objectives, along with other benefits, reside in the details of the construction and operation of the pet sanitation and waste management system as described in the claims.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF DRAWINGS

A more complete understanding of the present invention may be derived by referring to the detailed description when considered in connection with the following illustrative figures. In the figures, like reference numbers refer to like elements or acts throughout the figures.

FIG. 1 shows an isometric view of the pet sanitation and waste management system in accordance to one or more embodiments;

FIG. 2 shows a front view of pet sanitation and waste management system in accordance to one or more embodiments;

FIG. 3 shows a top view of the pet sanitation and waste management system in accordance to one or more embodiments;

FIG. 4 shows a side view of the pet sanitation and waste management system in accordance to one or more embodiments;

FIG. 5 shows a bottom view of the pet sanitation and waste management system in accordance to one or more embodiments;

FIG. 6 shows a back view of the pet sanitation and waste management system in accordance to one or more embodiments;

FIG. 7 shows an isometric exploded view of the pet sanitation and waste management system in accordance to one or more embodiments;

FIG. 8 shows an isometric exploded view of the pet sanitation and waste management system in accordance to one or more embodiments;

FIG. 9 shows a front isometric exploded view of the pet sanitation and waste management system in accordance to one or more embodiments;

FIG. 10 shows a side isometric exploded view of the pet sanitation and waste management system in accordance to one or more embodiments;

FIG. 11a shows a top exploded view omitting the containers of the pet sanitation and waste management system in accordance to one or more embodiments;

FIG. 11b shows a cross-sectional view of FIG. 11a of the pet sanitation and waste management system in accordance to one or more embodiments;

FIG. 12 shows a side isometric exploded view of the pet sanitation and waste management system in accordance to one or more embodiments;

FIG. 13 shows a front isometric exploded view of the pet sanitation and waste management system in accordance to one or more embodiments;

FIG. 14 shows a front exploded view omitting the containers of the pet sanitation and waste management system in accordance to one or more embodiments; and

FIG. 15 shows an side exploded view omitting the containers of the pet sanitation and waste management system in accordance to one or more embodiments.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

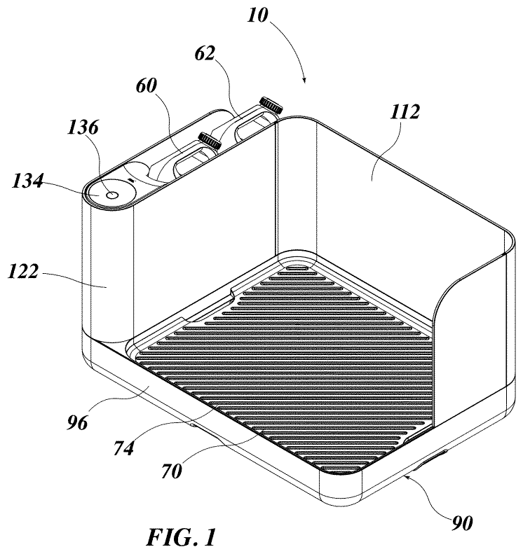

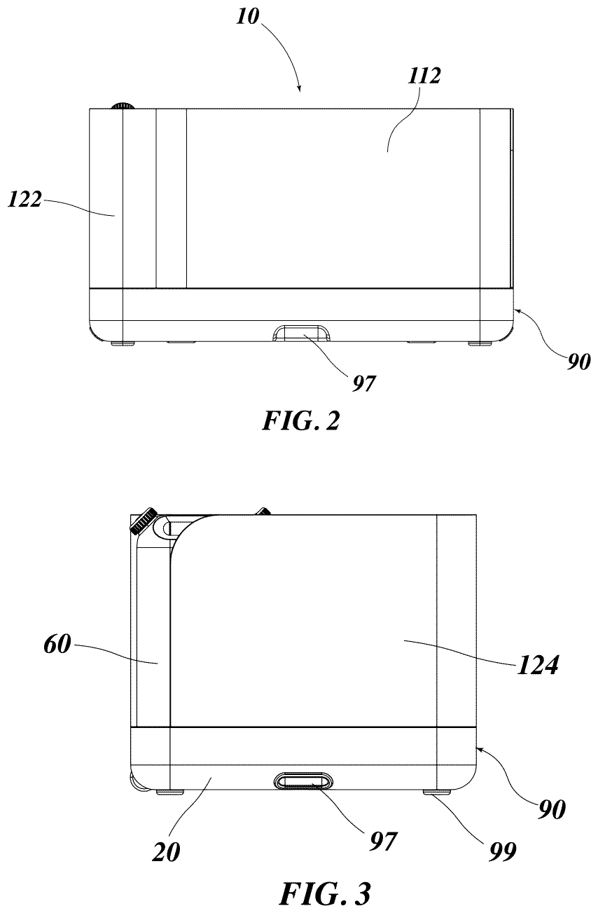

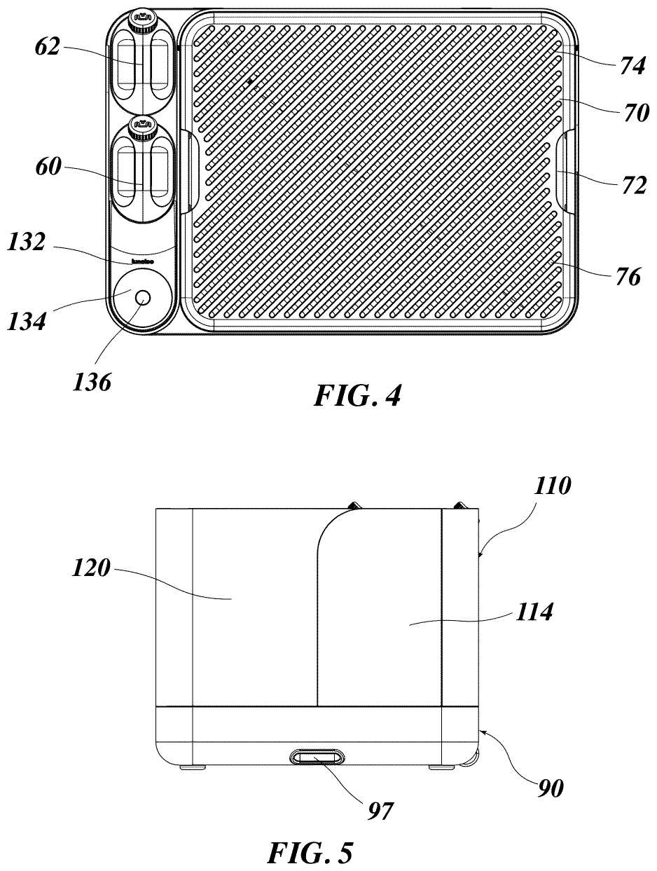

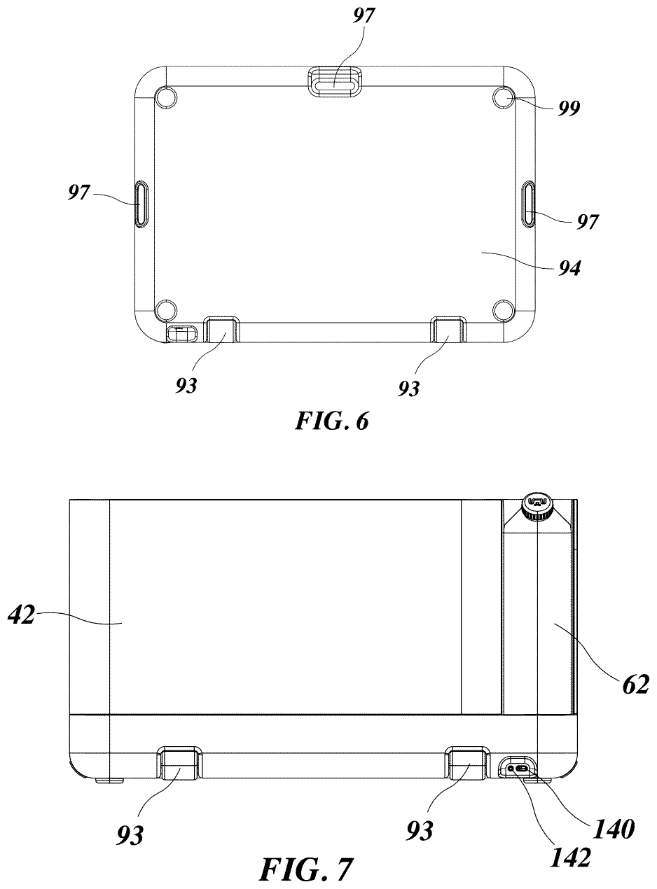

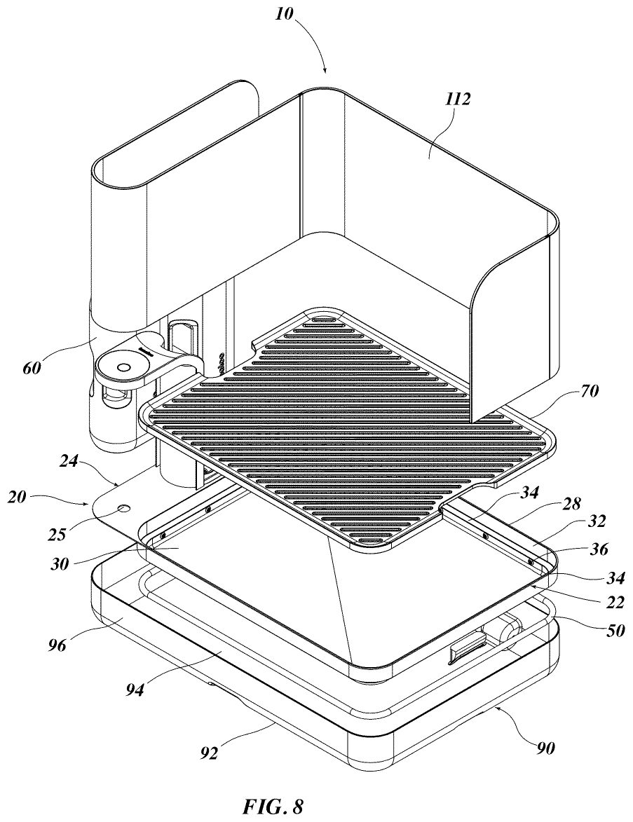

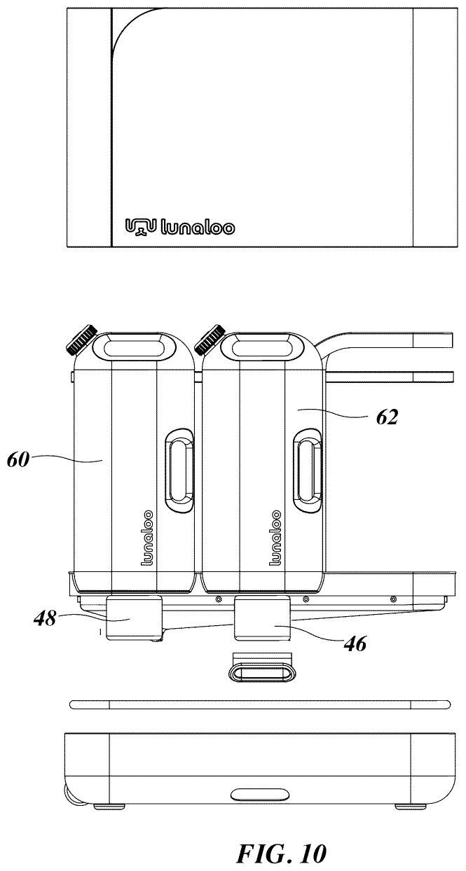

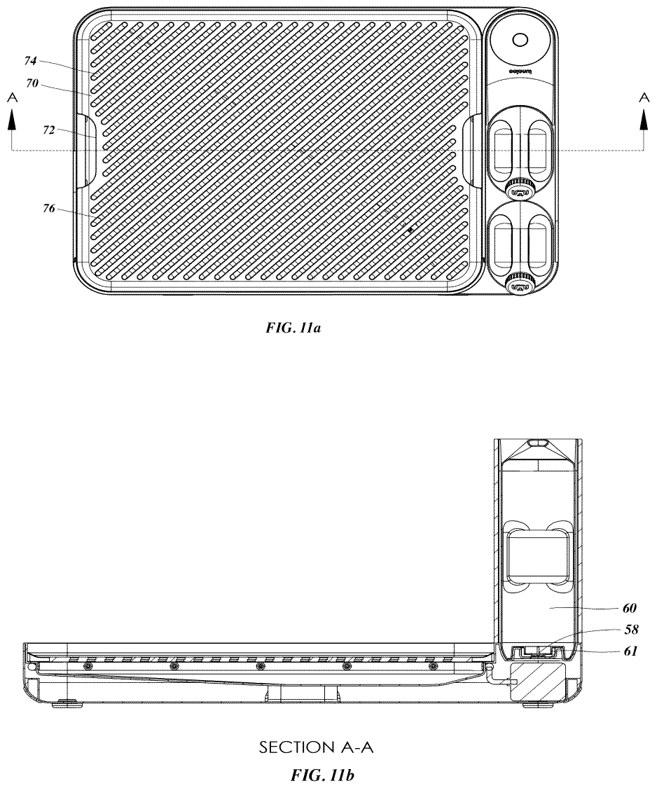

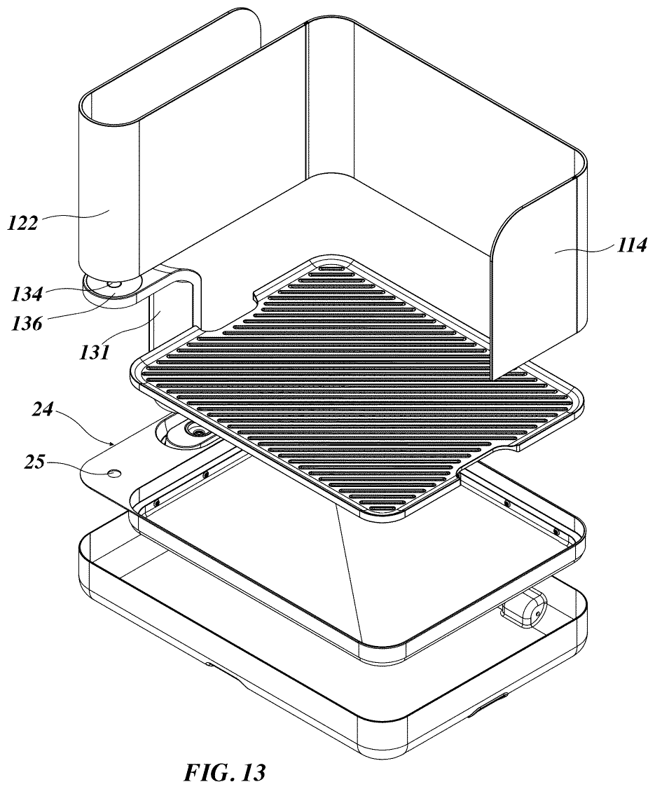

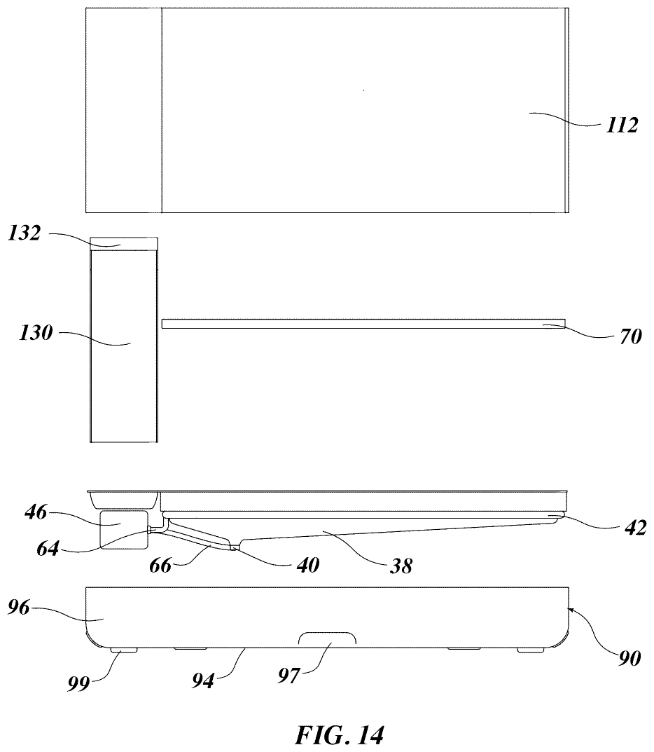

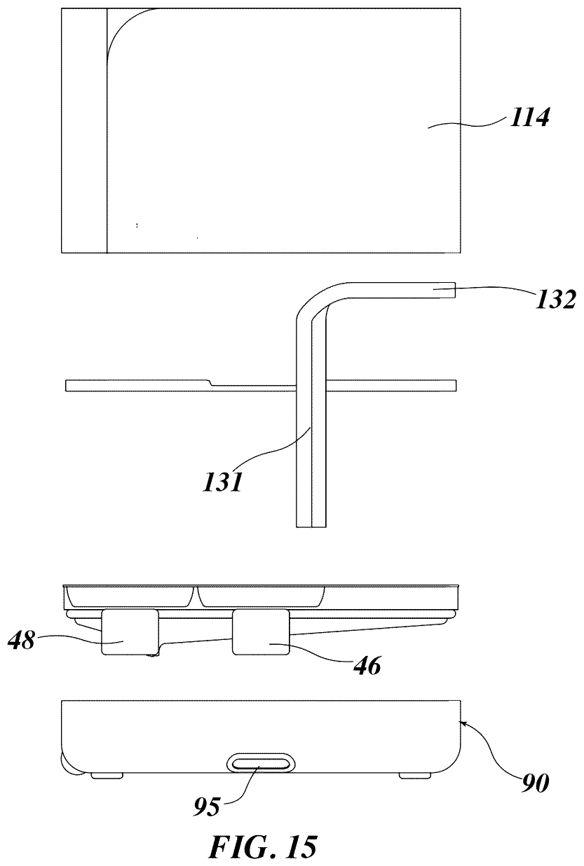

Referring initially to FIG. 1-15 a pet sanitation and waste management system is shown generally at 10. The pet sanitation and waste management system 10 can comprise a housing assembly wherein the housing assembly can have an inner housing 20 and an outer housing 90. The inner housing 20 can have a first inner section 22 and a second inner section 24. The first inner section 22 can include a pan 28 with a pan base 30, a pan inner perimeter 32, and a pan outer perimeter 34. The pan inner perimeter 32 can have a grate support 34 and a plurality of nozzles 36 located below the grate support. The grate support 34 can be a shelf within the pan inner perimeter 32 or in other embodiments the grate support can be such as, for example, flange, lip, rails, brackets, height posts, crossbars, grooves, slots, or the like wherein the grate support can support grate 70 which can be removably coupled to it. The grate support 34 can be integrated within the pan inner perimeter 32 or it can be separate and coupled to the pan inner perimeter. The grate 70 can be removed from the grate support for the user to easily clean. The grate 70 can have a grate handle 72 on at least one of its sides. The grate 70 can have a plurality of channels 74 and a plurality of holes 76 allowing the pet's waste to easily flow through the plurality of holes. The pan 28 and grate 70 collectively form a collection surface configured to receive the pet's waste. The grate 70 can be such as, for example, metal, stainless steel, aluminum, ceramic, composite, or the like and can be coated with rubber, silver-based coating, hydrophobic coating, chitosan coating, ceramic-based coating or the like wherein the coating can be anti-bacterial and provide a non-slip surface for the pet to step on. The grate 70 can be removed from the grate support 34 and easily cleaned by the user.

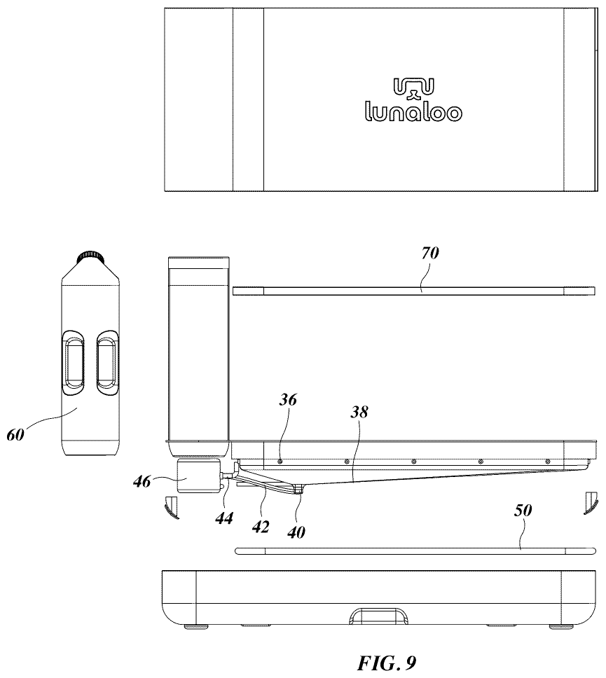

In embodiments, the plurality of nozzles 36 can be evenly or unevenly distributed on the pan inner perimeter 32. The plurality of nozzles 36 can be such as, for example, flat fan nozzles, full cone nozzles, hollow cone nozzles, jet nozzles, misting nozzles, spray nozzles, or the like. The plurality of nozzles 36 can be directed to spray a wash solution on at least one of the grate 70 and pan base 30. The pan base 30 can have an angled surface 38 configured to direct pet's waste towards a drain 40 when subjected to a flow of a cleaning fluid, as shown in FIG. 9. The angled surface 38 can be such as, for example, a single angled, a multi-slope angled, a contoured base, a recessed channel base or the like directing the pet's waste towards the drain 40. The plurality of nozzles 36 can be coupled to a wash tube 50 wherein the wash tube can surround the pan outer perimeter 34. The plurality of nozzles 36 can be coupled to the wash tube 50 by such as, for example, threaded coupling, compression fitting, push-to-connect, barbed coupling, clamp connection, welded connection, adhesive or the like. The wash tube 50 can be such as, for example, stainless steel, aluminum, brass, polyvinyl chloride, nylon, silicone, or the like. In certain embodiments the plurality of nozzles 36 can be integrated within the wash tube 50 and coupled to the pan outer perimeter 34 having a plurality of nozzle ports.

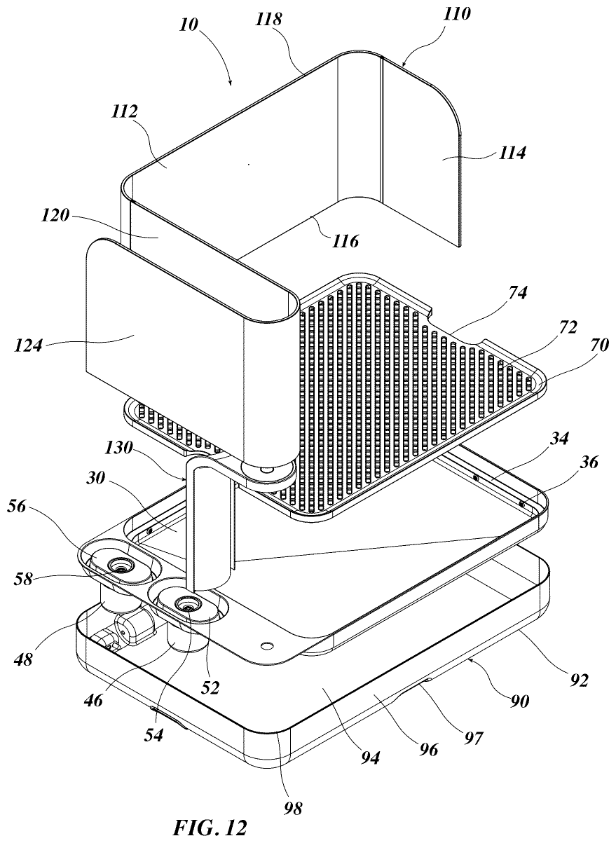

In embodiments, the second inner section 24 can include a waste receptacle 56 and a wash receptacle 52, both designed to be removably coupled to a waste container 60 and a wash container 62. The waste receptacle 56 and wash receptacle 52 can be shaped, as illustrated in FIG. 12, to accommodate the waste container 60 and wash container 62. The containers feature complementary shapes, enabling them to securely sit within their respective receptacles for stability and ease of use and allowing only one type of container to sit within the receptacles. The waste receptacle 56 and the wash receptacle 52 can have a waste valve 58 and a wash valve 54 coupled to or integrated within, wherein the waste valve and wash valve can be push open by the waste container 60 and the wash container 62 when removably coupled or in other embodiments, the waste container and the wash container can have the valve coupled to the bottle cap.

In embodiments, the waste valve 58 and wash valve 54 can be such as, for example, spring-loaded check valve, push-to-open valve, duckbill valve, flapper valve, plunger valve, or the like or in other embodiments the valve can be omitted. The waste valve 58 and wash valve 54 can be coupled to a waste motor 48 and a wash motor 46 wherein the waste motor and the wash motor can be such as, for example, vacuum pump, ejector pump motor, diaphragm pump motor, screw motor pump, combination pump system, variable-speed motors, brushless DC motors, or the like. The waste container 60 and the wash container 62 can have a twist on bottle cap 61 which can be sealed against waste receptacle 56 and wash receptacle 52 and the waste valve 58 and the wash valve 54 wherein when the waste container and the wash container are placed against the waste receptacle and the wash receptacle the waste valve 58 and a wash valve 54 is open allowing fluids to flow into the waste container and out of the wash container and when removed the valve can close the opening on the bottle cap on the waste receptacle and the wash receptacle.

In embodiments, the wash motor 46 can pull the wash fluid from the wash container 62 and push through the wash connection tube 44 to the wash tube 50 through the plurality of nozzles spraying the pan base 30. The wash motor 46, wash receptacle 52, wash tube 50, wash connection tube 44, and plurality of nozzles 36 collectively form a cleaning system configured to clean the collection surface. The wash motor 46 can create enough back pressure on the nozzles that each nozzle sprays substantially the same amount of wash fluid. In certain embodiments, the user can set the nozzle's direction and spray path.

In embodiments, the wash connection tube 44 can be made from the same or similar material as the wash tube 50. The wash fluid within the wash container 62 can be such as, for example, enzyme-based cleaner, vinegar-based solution, baking soda solution, hydrogen peroxide solutions, commercial pet-specific odor neutralizers, ammonia-free household cleaners, essential oils or the like. The waste container 60 and the wash container 62 can be such as, for example, plastic bottles, plastic containers, jugs, or the like and can be removably coupled to the waste receptacle 56 and the wash receptacle 52 wherein the containers can have a seating cap with one-way valve in its bottom allowing the fluid to flow through or allowing the waste to be deposited within the waste container.

The waste motor 48 can be coupled to a waste tube 42 which can be coupled to the drain 40 wherein the waste motor can pull the pet's waste and the cleaning fluid from the pan base 30 and fill the waste container 60 for the user to easily dispose of the pet's waste. The waste motor 48, waste receptacle 56, waste tube 42, and drain 40 collectively form a waste removal system configured to remove the pet's waste from the collection surface. The angled surface 38 can allow the cleaning fluid and pet's waste to be directed towards the drain 40 allowing the waste motor 48 to pull the combination of the cleaning fluid and pet's waste to pump into the waste container 60. The waste tube 42 can be coupled to the drain 40 on one end and the waste motor 48 on the other end by such as, for example, threaded coupling, compression fitting, push-to-connect, barbed coupling, clamp connection, welded connection, adhesive or the like.

In embodiments, the pet sanitation and waste management system 10 can further comprise an outer housing 90 coupled to the inner housing 24 and a guard 110. The outer housing 90 can have an open end 98 and closed end 92. The closed end 92 can form an outer housing base 94 wherein the closed end can be surrounded by one or more walls 96 extending from the perimeter of the closed end towards the open end, the walls can be configured to provide structural support and stability for accommodating the inner housing 24 wherein the guard can partially surround the one or more walls 96. The outer housing base 94 can have at least one foot 99 which can be rubber, plastic or the like and can provide anti-slip and cushion to the system. The outer housing 90 can have at least one wheel 93 rotatably coupled to it and at least one base handle 97 on at least one of the walls 96 allowing the user to easily grab and move the pet sanitation and waste management system 10. The inner housing 24 can be permanently or removably coupled to the outer housing 90 by such as, for example, adhesive, bolts, rivets, weld, press fit, thermal bonding, or the like. The inner housing 24 and the outer housing 90 can be manufactured from at least one of such as, for example, plastic, stainless steel, composite, ceramic, vinyl, or the like.

In embodiments, the guard 110 can extend from a guard lower end 116 to a guard upper end 118 wherein the guard lower end can be removably or permanently coupled to the open end 98 and/or the first inner section 22 and/or the second inner section 24 by such as, for example, adhesive, bolts, rivets, weld, press fit, thermal bonding, or the like. The guard 110 can provide protection from any pet's waste that may splash up from the grate 70 or from the animal missing the grate. The guard 110 can comprise a back panel 112 that transitions into a first side panel 114 and a second side panel 120 having a panel radius 122 that transitions into third side panel 124 each having the guard upper end 118 and the guard lower end 116. The second side panel 120, the panel radius 122 and the third side panel 124 can be a guide and support to the waste container 60 and the wash container 62 and can support and can be coupled to a power support 130.

The power support 130 can have a vertical member 131 and a horizontal member 132 wherein the vertical member extends from the second inner section 24 and the horizontal member can extend from the vertical member towards the panel radius 122. The horizontal member 132 can be coupled to a button 136 and an indication ring 134 wherein the horizontal member, button and indication ring can be substantially flush with the guard upper end 118. The button 136 and indication ring 134 can be one part or multiple parts such as, for example, push button with LED ring, touch button with LED ring, illuminated toggle button, keypad, smart button, or the like. When the button 136 is pushed the wash fluid is pumped from the wash container 62 and pumped through the wash tube 50 through the plurality of nozzles 36 spraying the pan base 30 flushing the pet's waste down the drain 40. The button 136 can have a cord that connects it to a power source or MCU which can be routed through a hole 25 on the second inner section 24. The back panel 112 can be made from such as, for example, plastic, stainless steel, aluminum, composite, or the like.

The pet's waste can be pumped through the waste tube 42 into the waste container 60. In certain embodiments, the system 10 can include one or more sensors, such as passive infrared sensors, pressure sensors, LiDAR sensors, optical sensors, accelerometers, vibration sensors, moisture sensors, wetness sensors, electrochemical sensors, pH sensors, thermal sensors, or similar devices. These sensors can be coupled to one or more components of the system, including the grate 70, the inner housing 20, the outer housing 90, and/or the guard 110. The sensors detect when an animal steps onto the grate, relieves itself, and exits the grate. Upon detecting the animal's exit, the system automatically initiates a flush sequence as described in the steps above. The sensors can be connected to a microcontroller (“MCU”) and powered by a power source linked to an on/off switch 140 and a power adapter port 142, which may be integrated into the outer housing 90. The MCU, sensors, button 136, and associated control electronics collectively form a control system configured to activate the waste removal system and the cleaning system.

In some embodiments, the system may include a wireless communication module and a camera, enabling the user to receive notifications when the pet relieves itself and view an image of the pet. The wireless communication module can comprise such as, for example, a Wi-Fi module, Bluetooth module, cellular module, or combinations thereof, and is configured to communicate with a remote computing device such as a smartphone, tablet, or computer. In certain embodiments, a camera system which can include a camera, speaker and microphone can be coupled to the guard 110 allowing the user to see, talk to, and listen to the pet while it is on the system wherein the camera, speaker and microphone can be one system or separate pieces. The camera, microphone, and speaker are configured to enable two-way audio and video communication between the user and the pet. The user can use an application executing on the remote computing device to view live video from the camera, speak to the pet through the speaker, and hear sounds through the microphone. This two-way communication allows the user to monitor the pet remotely, provide comfort or commands, and observe the pet's behavior while away from home. The camera system can be wirelessly coupled to a portable computing device or computer via WiFi or Bluetooth.

The wireless communication module enables the system to connect to a mobile application or web-based application executing on the remote computing device. The application provides a user interface for controlling the system remotely, including the ability to trigger the cleaning system and waste removal system through remote commands sent from the application to the control system. When the user activates a remote flushing command in the application, the wireless communication module receives the command and transmits it to the control system, which then activates the wash motor 46 and waste motor 48 to perform a cleaning cycle. The application displays real-time system status, notifications of waste collection events, weight tracking data presented in graphs and charts, urinalysis results and historical trends, health alerts when potential conditions are detected, and access to live camera feed and two-way communication features. The user can review historical data, set up automated cleaning schedules, receive push notifications on their mobile device, and manage all system settings through the application. The control system can be configured to transmit data to the application comprising system status information, waste collection event timestamps, pet monitoring information including images and video clips, weight measurements, and urinalysis data.

In embodiments, the system 10 can include a weight sensor configured to measure the pet's weight when the pet is positioned on the collection surface. The weight sensor can comprise a load cell, strain gauge, pressure sensor, force transducer, or similar weight measurement device positioned beneath the pan 28, integrated into the grate support 34, or positioned between the pan 28 and the first inner section 22. The weight sensor can be a single sensor or an array of multiple sensors positioned to accurately measure the pet's weight regardless of the pet's position on the collection surface. The weight sensor can be coupled to the control system and configured to transmit weight measurements to the MCU. The control system can receive the weight measurement signal, processes the data to calculate the pet's weight, and stores each weight measurement with a timestamp in memory, creating a historical record of the pet's weight over time. The control system can track weight changes by comparing current measurements to previous measurements, calculate trends such as weight gain or loss rates, and generate alerts to the user when significant weight changes occur that may indicate health concerns. For example, the control system can alert the user if the pet loses more than a predetermined percentage of body weight over a specified time period, or if the pet gains weight at an excessive rate. Weight data can be transmitted to the application on the remote computing device for user review and tracking, where the application can display weight trends in graphical format, compare weight against target ranges, and provide insights into the pet's health status.

In embodiments, the system 10 can include one or more urinalysis sensors positioned to contact the pet's urine on the collection surface. The urinalysis sensors can comprise electrochemical sensors, pH sensors, optical sensors, colorimetric test strip readers, spectrophotometric sensors, impedance sensors, or combinations thereof. The sensors can be positioned in the pan 28, integrated into the grate 70, embedded in the pan base 30, or positioned adjacent to the drain 40 to ensure contact with the pet's urine during urination. In some embodiments, the urinalysis sensors can be removable for cleaning or replacement. The urinalysis sensors are configured to detect various urinary parameters including pH level, glucose concentration, protein levels, presence of blood (hematuria), ketone levels, specific gravity, bilirubin levels, urobilinogen levels, nitrite levels, leukocyte levels, and other biomarkers indicative of health conditions.

Each urinary parameter provides valuable health information. The pH level measurement indicates the acidity or alkalinity of the urine, with normal ranges typically between 6.0 and 7.5 for most pets; abnormal pH levels may indicate urinary tract infections, kidney stones, or metabolic disorders. Glucose detection identifies the presence of sugar in the urine, which may indicate diabetes mellitus when glucose is present at elevated levels. Protein level measurement detects the presence of protein in urine, which may suggest kidney dysfunction, urinary tract infection, or other kidney-related conditions when protein levels are abnormally high. Blood presence detection identifies red blood cells in the urine (hematuria), which may indicate urinary tract infection, bladder stones, kidney disease, or trauma. Ketone level measurement detects ketones in the urine, which may indicate diabetes, starvation, or metabolic disorders. Specific gravity measurement indicates the concentration of dissolved substances in the urine and provides information about kidney function and hydration status, with abnormal specific gravity potentially indicating kidney disease or dehydration.

The control system is configured to receive sensor data from the urinalysis sensors and analyze the urinary parameters to detect potential health conditions. The control system can include stored algorithms, lookup tables, or artificial intelligence models that compare measured urinary parameters against normal ranges and identify patterns indicative of specific health conditions. For example, the control system can detect urinary tract infection based on a combination of abnormal pH levels, presence of nitrites, elevated leukocyte levels, and presence of blood. The control system can detect kidney dysfunction based on elevated protein levels, abnormal specific gravity, and presence of blood. The control system can detect diabetes based on presence of glucose and ketones in the urine. The control system can detect hematuria (blood in urine) based on blood presence detected by the sensors, which may indicate various conditions requiring veterinary attention. The control system stores the urinary parameter data in memory along with timestamps and tracks trends over time, comparing current measurements against baseline values established during initial use and against normal ranges stored in the system. By tracking trends, the control system can identify gradual changes that may indicate developing health issues before they become severe.

When the control system detects abnormal urinary parameters or identifies a potential health condition based on its analysis, it generates a health alert. The health alert can include information about which parameters are abnormal, the severity of the abnormality, the potential health condition detected, and recommendations for action such as consulting a veterinarian. The wireless communication module transmits this health alert to the application on the remote computing device, where the user receives a notification such as a push notification, email, or in-app alert. The notification notifies the user of the detected condition and can recommend veterinary consultation or further monitoring. The system maintains a historical log of all urinalysis data in the control system's memory and/or on cloud storage accessible through the application, enabling the user to review trends over days, weeks, or months. The user can view detailed urinalysis reports in the application, showing measurements for each parameter over time presented in tables, graphs, or charts. This historical data can be exported or shared with veterinary professionals to aid in diagnosis and treatment. The application can generate reports summarizing the pet's health trends, highlighting any concerning patterns, and tracking the effectiveness of treatments or dietary changes over time.

In operation, when a pet steps onto the collection surface, the weight sensor measures the pet's weight and the control system records the measurement with a timestamp. The sensors may also detect the pet's presence through motion detection, pressure changes, or other sensing methods. As the pet urinates on the collection surface, the urine flows through the plurality of holes 76 in the grate 70 and contacts the urinalysis sensors positioned in or near the pan 28. The urinalysis sensors analyze the urine in real-time or near real-time and detect the various urinary parameters described above. The sensor data is transmitted to the control system, which analyzes the parameters and compares them against normal ranges and stored baseline data to detect potential health conditions. If a potential health condition is detected based on abnormal urinary parameters, the control system generates a health alert and transmits it to the user's remote computing device via the wireless communication module, notifying the user of the detected condition.

After the pet exits the collection surface, which can be detected by the sensors monitoring the collection surface, the system can automatically initiate a cleaning cycle or wait for manual activation via the button 136 or a remote command from the application. When a cleaning cycle is initiated, either automatically, manually via button press, or remotely via app command, the control system activates the cleaning system by engaging the wash motor 46. The wash motor 46 pulls wash fluid from the wash container 62 through the wash connection tube 44 and pumps it through the wash tube 50 to the plurality of nozzles 36, which spray the wash fluid onto the collection surface including the grate 70 and pan base 30. The wash fluid cleans and sanitizes the collection surface. Simultaneously or subsequently, the control system activates the waste removal system by engaging the waste motor 48, which creates suction that pulls the waste, urine, and wash fluid from the pan base 30 through the drain 40 and waste tube 42 into the waste container 60 for disposal. The angled surface 38 of the pan base 30 directs all liquid toward the drain 40 to ensure complete waste removal.

Throughout the process described above, the camera can capture images or video of the pet for monitoring purposes, documentation of health events, or simply for the user' viewing pleasure. The weight data, urinalysis data, timestamps, and images are all stored in the control system's memory and transmitted to the application on the remote computing device for user review. The application provides a comprehensive interface where the user can view current and historical weight data presented in graphs showing weight trends over time, review urinalysis results with color-coded indicators for normal and abnormal parameters, receive health alerts with detailed information about detected conditions, view captured images and videos of the pet, control the system remotely by triggering cleaning cycles, communicate with the pet via two-way audio and video, and configure system settings such as automatic cleaning schedules and alert thresholds. This integrated monitoring system provides the user with comprehensive health tracking capabilities, enabling early detection of potential health issues and allowing the user to provide better care for their pet.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

I claim:

1. A pet sanitation and waste management system for collecting and disposing of waste from a pet, comprising:
a housing assembly;
a collection surface having an access by which the pet may access the collection surface, wherein the collection surface is disposed in the housing assembly and configured to receive the waste;
a waste removal system coupled to the collection surface and configured to remove the pet's waste from the collection surface; and
a cleaning system configured to clean the collection surface; and
a control coupled to the waste removal system and the cleaning system and configured to selectively activate the waste removable system and the cleaning system.

2. The system of claim 1, wherein the housing assembly comprises an inner housing and an outer housing, the collection surface comprises a pan having an angled base configured to direct the pet's waste towards a drain and a support grate removably positioned above the pan, the cleaning system comprises a plurality of nozzles configured to spray the pan, and the waste removal system comprises a waste pump coupled to the drain.

3. The system of claim 1, wherein the support grate has a plurality of holes configured to allow the pet's waste to flow therethrough and having an antibacterial and anti-slip coating.

4. The system of claim 1, wherein the cleaning system further comprises a wash receptacle configured to couple to a removable wash container, and the waste removal system further comprises a waste receptacle configured to couple to a removable waste container.

5. The system of claim 1, wherein the housing assembly includes at least two wheels and at least one handle.

6. The system of claim 1, further comprising a weight sensor coupled to the collection surface and configured to measure a weight of the pet, wherein the control system is configured to store a plurality of weight measurements over time and track weight changes.

7. The system of claim 1, further comprising a camera, a microphone, and a speaker coupled to the housing assembly and configured to enable two-way audio and video communication between a user and the pet.

8. The system of claim 1, further comprising a wireless communication module configured to communicate with a remote computing device executing an application.

9. The system of claim 8, wherein the control system is configured to receive commands from the application to remotely activate the waste removal system and the cleaning system, and to transmit data to the application comprising at least one of system status, waste collection events, or pet monitoring information.

10. The system of claim 1, further comprising at least one sensor coupled to the control and configured to analyze the pet's urine and detect at least one urinary parameter selected from the group consisting of pH level, glucose level, protein level, blood presence, ketone level, and specific gravity.

11. The system of claim 10, wherein the control is configured to analyze the at least one urinary parameter and detect potential health conditions comprising at least one of urinary tract infection, kidney dysfunction, diabetes, or hematuria.

12. The system of claim 11, further comprising a wireless communication module configured to transmit a health alert to an application on a remote computing device when the control system detects a potential health condition.

13. The system of claim 11, wherein the control system is configured to store historical data of the at least one urinary parameter and track trends over time.

14. The system of claim 6, further comprising at least one sensor configured to analyze the pet's urine, a camera configured to capture images of the pet, and a wireless communication module configured to transmit weight data, urinalysis data, and images to an application on a remote computing device.

15. The system of claim 14, wherein the application is configured to provide remote flushing control, two-way communication with the pet, weight tracking data, urinalysis tracking data, and health monitoring alerts.

16. A method of monitoring pet health using a sanitation system, comprising:
receiving a pet on a collection surface of the sanitation system;
measuring a weight of the pet using a weight sensor;
analyzing urine from the pet using at least one sensor to detect at least one urinary parameter;
detecting a potential health condition based on the at least one urinary parameter;
transmitting a health alert to a remote computing device when the potential health condition is detected; and
removing the urine from the collection surface using a waste removal system.

17. The method of claim 16, further comprising receiving a remote command from an application on the remote computing device and activating the waste removal system in response to the remote command.

18. The method of claim 16, further comprising storing historical data of the weight and the at least one urinary parameter, and tracking trends over time to identify changes in pet health.

19. The method of claim 16, further comprising capturing images of the pet using a camera and enabling two-way audio and video communication between a user and the pet via a microphone and speaker.
